(19) United States
(12) Patent Application Publication
(10) Pub. No.: US 2024/0341720 A1
(43) Pub. Date: Oct. 17, 2024

(54) PUNCTURE GUIDING APPARATUS AND ULTRASONIC DIAGNOSTIC SYSTEM

(71) Applicant: Canon Medical Systems Corporation, Otawara-shi (JP)

(72) Inventors: Shohei SASAKI, Otawara (JP); Yasuhisa MAKITA, Nasushiobara (JP); Hana SONODA, Utsunomiya (JP)

(73) Assignee: Canon Medical Systems Corporation, Otawara-shi (JP)

(21) Appl. No.: 18/628,870

(22) Filed: Apr. 8, 2024

(30) Foreign Application Priority Data
Apr. 11, 2023 (JP) 2023-064208

(51) Int. Cl. A61B 8/08; A61B 8/00; A61B 8/06; A61B 34/10; A61B 34/20; A61B 90/00

(52) U.S. Cl. CPC A61B 8/0841; A61B 8/06; A61B 8/4488; A61B 8/488; A61B 8/5246; A61B 34/10; A61B 34/20; A61B 90/08; A61B 2034/107; A61B 2034/2057; A61B 2034/2063; A61B 2090/0811

(57) ABSTRACT

A puncture guiding apparatus includes a detector and processing circuitry. The detector is configured to detect passage of a puncture needle, the detector arranged in a vicinity of a living body contacting surface of an ultrasonic probe that is brought into contact with a living body. The processing circuitry is configured to output identification information for identifying a positional relationship between the puncture needle and a scanning plane of the ultrasonic probe based on a detection result obtained by the detector.

### CROSS-REFERENCE TO RELATED APPLICATION

This application is based upon and claims the benefit of priority from Japanese Patent Application No. 2023-064208, filed Apr. 11, 2023, the entire contents of which are incorporated herein by reference.

### FIELD

Embodiments described herein relate generally to a puncture guiding apparatus and an ultrasonic diagnostic system.

### BACKGROUND

An ultrasonic diagnostic system emits ultrasound waves on a subject by an ultrasonic probe in which a plurality of ultrasonic transducers are aligned, and generates an ultrasound image through receiving reflected ultrasound waves with the ultrasonic probe.

A puncture technique using an ultrasonic probe and a puncture needle may be performed for nerve blocks and venous access under ultrasonic guidance. With such a puncture technique, a technician operates an ultrasonic probe so that a target area is located in the scanning plane of the ultrasonic probe, and punctures the target area with the puncture needle, checking the target area on an ultrasound image. At this time, in order to puncture the target area accurately, the technician needs to puncture the target area with a puncture needle while the puncture needle is located in the plane including the scanning plane of the ultrasonic probe. If the target area is located in the plane perpendicular to the scanning plane of the ultrasonic probe, in order to puncture the target area accurately, the technician needs to puncture the target area while the puncture needle is located in the plane perpendicular to the scanning plane.

As techniques for puncturing a target area accurately, a puncture adapter having a puncture guide into which a puncture needle is inserted may be attached to an ultrasonic probe, or a ultrasonic probe having a puncture guideline marker may be used. In the case of using a puncture adapter, however, a puncture guide needs to be replaced depending on a diameter of the puncture needle. If the diameter of the puncture needle and the diameter of the puncture guide do not match, wobbling of the puncture needle may occur when the puncture needle is inserted into the puncture guide, and an accurate puncture may not be achieved. In the case of using a guideline marker, on the other hand, the marker, if provided in the vicinity of the living body contacting part of the ultrasonic probe, may be hidden behind a living body and difficult to find, and in turn an accurate puncture may not be achieved.

### BRIEF DESCRIPTION OF THE DRAWINGS

FIG. 1 is a diagram showing an example of a configuration of an ultrasonic diagnostic system according to a first embodiment.

FIG. 2 is a diagram of a puncture guiding apparatus according to the first embodiment, viewed from one side with respect to a short-axis direction.

FIG. 3 is a perspective view of the puncture guiding apparatus according to the first embodiment, viewed from one side with respect to a scanning direction.

FIG. 4 is a flowchart illustrating a processing procedure by the puncture guiding apparatus according to the first embodiment.

FIG. 5 is a diagram showing how a puncture is performed using the puncture guiding apparatus of the first embodiment, viewed from one side with respect to the short-axis direction.

FIG. 6 is a diagram showing in-plane alignment of a puncture needle shown in FIG. 5, viewed from one side with respect to the scanning direction.

FIG. 7 is a diagram showing out-of-plane alignment of the puncture needle shown in FIG. 5, viewed from one side with respect to the scanning direction.

FIG. 8 is a sectional view of a puncture guiding apparatus of a first modification of the first embodiment, in a section passing through the scanning plane of the puncture guiding apparatus.

FIG. 9 is a sectional view of a puncture guiding apparatus of a second modification of the first embodiment, in a section passing through the scanning plane of the puncture guiding apparatus.

FIG. 10 is a diagram showing an example of a configuration of an ultrasonic diagnostic system according a third modification of the first embodiment.

FIG. 11 is a diagram of a puncture guiding apparatus according to the third modification of the first embodiment, viewed from one side with respect to the short-axis direction.

FIG. 12 is a flowchart illustrating a processing procedure by the puncture guiding apparatus according to the third modification of the first embodiment.

FIG. 13 is a diagram showing an example of an ultrasound image displayed on a display in the third modification of the first embodiment.

FIG. 14 is a diagram of a puncture guiding apparatus according to the second embodiment, viewed from one side with respect to the short-axis direction.

FIG. 15 is a perspective view of the puncture guiding apparatus according to the second embodiment, viewed from one side with respect to the scanning direction.

FIG. 16 is a flowchart illustrating a processing procedure by the puncture guiding apparatus according to the second embodiment.

FIG. 17 is a diagram showing how a puncture is performed using the puncture guiding apparatus of the second embodiment, viewed from one side with respect to the scanning direction.

FIG. 18 is a diagram showing in-plane alignment of a puncture needle shown in FIG. 17, viewed from one side with respect to the short-axis direction.

FIG. 19 is a diagram showing out-of-plane alignment of the puncture needle shown in FIG. 17, viewed from one side with respect to the short-axis direction.

### DETAILED DESCRIPTION

In general, according to one embodiment, a puncture guiding apparatus includes a detector and processing circuitry. The detector is configured to detect passage of a puncture needle, the detector arranged in a vicinity of a living body contacting surface of an ultrasonic probe that is brought into contact with a living body. The processing circuitry is configured to output identification information for identifying a positional relationship between the puncture needle and a scanning plane of the ultrasonic probe based on a detection result obtained by the detector.

Hereinafter, the embodiments of a puncture guiding apparatus and an ultrasonic diagnostic system will be explained in detail with reference to the accompanying drawings. In the description hereinafter, structural elements having substantially the same functions and configurations will be denoted by the same reference symbols, and a duplicate description of such elements will be given only where necessary.

### First Embodiment

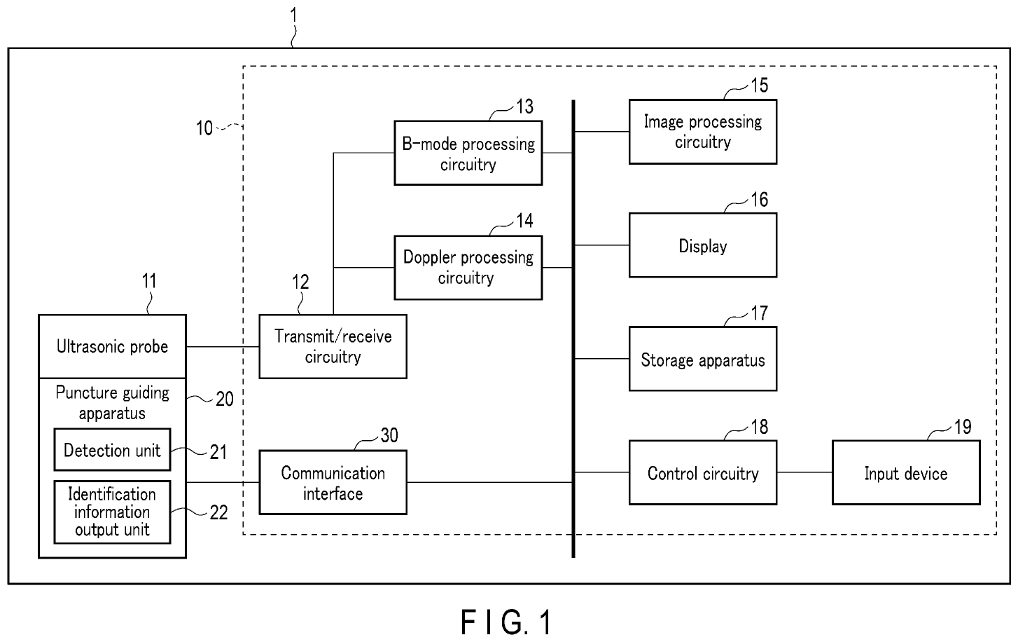

FIG. 1 is a diagram showing an example configuration of an ultrasonic diagnostic system 1 that includes a puncture guiding apparatus 20 according to the first embodiment. The ultrasonic diagnostic system 1 of the present embodiment is used with a technique of inserting a puncture needle in a scanning direction (long-axis direction) of an ultrasonic probe 11. As shown in FIG. 1, the ultrasonic diagnostic system 1 includes an ultrasonic probe 11, transmit/receive circuitry 12, B-mode processing circuitry 13, Doppler processing circuitry 14, image processing circuitry 15, a display 16, a storage apparatus 17, control circuitry 18, an input device 19, a puncture guiding apparatus 20, and a communication interface 30. The ultrasonic diagnostic system 1 may be called an “ultrasonic diagnostic apparatus”. The transmit/receive circuitry 12, the B-mode processing circuitry 13, the Doppler processing circuitry 14, the image processing circuitry 15, the display 16, the storage apparatus 17, the control circuitry 18, the input device 19, and the communication interface 30 are packaged in a single housing that may be called an “apparatus main body 10”. The ultrasonic probe 11 is detachably attached to the apparatus main body 10 via a cable. The puncture guiding apparatus 20 is detachably connected to the ultrasonic probe 11, and can communicate with the communication interface 30 of the apparatus main body 10, by wire or wirelessly.

The ultrasonic probe 11 is a device (probe) that takes charge of transmitting and receiving ultrasound waves emitted from and reflected on a subject, and consists of an electric/mechanical reversible sensing element. The ultrasonic probe 11 is composed of, for example, a phased-array type probe whose distal end is equipped with a plurality of elements arranged in an array. This allows the ultrasonic probe 11 to convert a pulse drive voltage of a supplied driving signal into an ultrasonic pulse signal and transmit it in a desired direction within a scan region of a subject (hereinafter, the direction will be called an “acoustic radiation direction”) and to convert the ultrasonic signal reflected from the subject into an echo signal of a corresponding voltage.

For the ultrasonic signal transmission, the transmit/receive circuitry 12 supplies a driving signal to the ultrasonic probe 11. Specifically, the transmit/receive circuitry 12 has trigger generating circuitry, delay circuitry, pulser circuitry, and the like. The pulser circuitry repeatedly generates rate pulses for forming transmission ultrasound waves at a predetermined rate frequency. The delay circuitry provides each rate pulse generated by the pulser circuitry with a delay time for each piezoelectric oscillator, which is necessary for converging ultrasound generated by the ultrasonic probe 11 into a beam form and determining transmission directivity. The trigger generating circuitry supplies driving signals (driving pulses) to the ultrasonic probe 11 at a timing based on the rate pulse. In other words, by varying the delay time provided to each rate pulse, the delay circuitry adjusts a direction of a transmission from the piezoelectric oscillator surface as appropriate.

The transmit/receive circuitry 12 has a function of changing a transmit frequency and a transmit drive voltage, etc. instantaneously based on an instruction from the control circuitry 18 so as to perform a predetermined scan sequence. Particularly, the change in a transmit drive voltage is realized by an origination circuit capable of instantaneous switching of the voltage value, or a mechanism for electrically switching a power source unit to another.

For the ultrasonic signal reception, the transmit/receive circuitry 12 executes various types of processes on the reflected echo signals in accordance with a reflected wave signal received by the ultrasonic probe 11 and converts the echo signal to reflected wave data in accordance with reception directivity. Specifically, the transmit/receive circuitry 12 has an amplifier circuit, an A/D converter, and an adder, etc. The amplification circuitry executes a gain correction processing for each channel by amplifying reflected wave signals. The A/D converter performs A/D conversion on a gain-corrected reflected wave signal and gives digital data a delay time required for determining reception directivity. The adder adds up A/D-converted reflected wave signals and generates reflected wave data. By the adding process of the adder, a reflected component is enhanced in a direction corresponding to the reception directivity of the reflected wave signal.

The B-mode processing circuitry 13 performs logarithmic amplification, envelope detection processing, and logarithmic compression, etc. on the reflected wave data from the transmit/receive circuitry 12 and generates B-mode information in which a signal strength at each sample point is expressed in a luminance level.

The Doppler processing circuitry 14 performs a color Doppler technique on the reflected wave data from the transmit/receive circuitry 12 and calculates blood flow information, namely Doppler information. With the color Doppler technique, the ultrasonic transmission and reception is performed on the same scanning line multiple times, and an MTI (moving target indicator) filter is applied to a same-positioned data column in order to inhibit signals (clutter signals) originating from a static tissue or slow-moving tissue and to extract signals originating from blood flow. Furthermore, with the color Doppler technique, Doppler information, such as a blood flow rate, blood flow dispersion, and blood flow power, etc., is estimated from these blood flow signals.

The image processing circuitry 15 is a processor performing image processing. The image processing circuitry 15 converts the scanning scheme of the B-mode information to a scanning scheme suitable for displaying (scanning conversion), and generates a B-mode image of a subject. Similarly, the image processing circuitry 15 performs scanning conversion of the scanning method of the Doppler information to a scanning method suitable for display and generates a Doppler image of a subject. Display images such as a B-mode image and a Doppler image will be collectively called “ultrasound images”. The image processing circuitry 15 also generates, together with the ultrasound images, information indicating compositing, parallel arrangement, or display positioning of each image information item, and various kinds of information used to assist the operation of the ultrasonic diagnostic system 1, and attendant information required for ultrasonic diagnosis such as patient information.

The display 16 is a display device that displays visual video information converted from display information provided from the image processing circuitry 15, in conjunction with the image processing circuitry 15. For example, the display 16 displays a fusion image generated by the image processing circuitry 15. As the display 16, a CRT display, a liquid crystal display, an organic EL display, and a plasma display are applicable for example. A projector may be provided as the display 16.

The storage apparatus 17 is a type of storage such as a ROM (read only memory), a RAM (random access memory), an HDD (Hard Disk Drive), an SSD (Solid State Drive), or an integrated circuit storage device, etc. which stores various types of information. The storage apparatus 17 may also be, for example, a drive that performs reading and writing various kinds of information on a portable storage medium such as a CD-ROM drive, a DVD drive, or a flash memory. For example, the storage apparatus 17 stores various types of information, such as B-mode information, Doppler information, a B-mode image, a Doppler image, and a fusion image, etc.

The storage apparatus 17 stores programs executed by the processing circuitry, and various types of data used for the processing in the processing circuitry. As the programs, programs that can be installed onto a computer from a network or a non-transitory computer readable storage medium and that cause the computer to realize the functions of processing circuitry are used. Various types of data used in the present description are typically digital data.

The control circuitry 18 is a processor that controls the entire processing in the ultrasonic diagnostic system 1. The control circuitry 18 executes a program stored in the storage apparatus 17 to realize a function corresponding to the program. Specifically, the control circuitry 18 controls the processing in the transmit/receive circuitry 12, the B-mode processing circuitry 13, the Doppler processing circuitry 14, and the image processing circuitry 15, based on various setting requests that are input by an operating person via an input device 19, various control programs, and various types of data. The control circuitry 18 has a display control function and outputs various types of information via the display 16. For example, the control circuitry 18 causes the display 16 to display an image generated by the image processing circuitry 15. The control circuitry 18 has a function to interface with the input device 19.

The input device 19 is various types of user interfaces on a touch panel or an operation panel. An operating person can input various operations and commands to the ultrasonic diagnostic system 1 via the input device 19. The display 16 and the input device 19 are not necessarily separated and they may be integrated as a mechanism.

The puncture guiding apparatus 20 is an external adapter detachably attached to the ultrasonic probe 11.

The communication interface 30 is connected to the puncture guiding apparatus 20 with wire or wirelessly for data communication. Hereinafter, the communication interface 30 will be omitted in the descriptions regarding communication between the apparatus main body 10 and the puncture guiding apparatus 20.

The puncture guiding apparatus 20 includes a detection unit (detector) 21 and an identification information output unit 22. In the present embodiment, an example is given wherein the puncture guiding apparatus 20 and the ultrasonic probe 11 are separate components; however, the puncture guiding apparatus 20 may be integrated with the ultrasonic probe 11. In the latter case, the detection unit 21 and the identification information output unit 22 are mounted in the ultrasonic probe 11.

The detection unit 21 is provided in the vicinity of the living body contacting surface of the ultrasonic probe 11, and detects passage of the puncture needle. The living body contacting surface is a contacting surface of the ultrasonic probe 11 that is brought into contact with a living body, and is arranged at the distal end of the ultrasonic probe 11, for example. For this reason, the detection unit 21 is provided in the vicinity of the distal end portion of the ultrasonic probe 11. The detection unit 21 is provided at a position at which the positional relationship between the puncture needle and the scanning plane of the ultrasonic probe 11 (scanning cross section) can be determined. For example, the detection unit 21 is arranged on a plane including the scanning plane, and detects passage of the puncture needle within the plane including the scanning plane.

The detection unit 21 is realized by a non-contact sensor that detects a presence/absence of the puncture needle within a predetermined detection range and a processor that analyzes a sensor signal by executing a program stored in a not-shown storage apparatus implemented in the puncture guiding apparatus 20. As the non-contact sensor, a camera, an optical sensor, or an ultrasonic sensor may be used, for example. An example of the optical sensor is an infrared sensor. The processing circuitry analyzes a signal obtained from the sensor to detect passage of the puncture needle within a detection range, and transmits the detection results to the identification information output unit 22. Hereinafter, the present embodiment will be described using an example wherein the detection unit 21 is provided with a camera.

The identification information output unit 22 outputs identification information for identifying a positional relationship between the puncture needle and the puncture plane including a target position at which the puncture needle is slated to be inserted based on a detection result of the detection unit 21. In the present embodiment, the puncture plane is a plane parallel to the scanning plane of the ultrasonic probe 11 and including the scanning plane. For this reason, the identification information output unit 22 outputs identification information for identifying the positional relationship between the puncture needle and the scanning plane of the ultrasonic probe 11. The identification information output unit 22 is realized by a processor that performs processing by executing a program stored in a not-shown storage apparatus implemented in the puncture guiding apparatus 20, for example. The identification information output unit 22 is described as a single apparatus performing a plurality of functions; however, the plurality of functions may be performed by separate apparatuses. For example, the functions of the identification information output unit 22 may be implemented on different apparatuses in a distributed manner.

More specifically, the identification information output unit 22 outputs identification information for identifying the puncture needle to be located in the plane including the scanning plane. The identification information output unit 22 determines whether the puncture needle is in or out of plane with the scanning plane based on the detection result obtained from the detection unit 21. If the puncture needle is in plane with the scanning plane, the entire shaft of the puncture needle is in parallel to the puncture plane and is located in the puncture plane. If the puncture needle is out of the scanning plane, it means that the puncture needle is tilted with respect to the puncture plane and at least a part of the puncture needle is located out of the puncture plane.

Thereafter, the identification information output unit 22 outputs, as the identification information, information regarding whether the puncture needle is in an in-plane state or an out-of-plane state with respect to the scanning plane, and notifies the user of the identification information. The user is for example a technician who performs a puncture technique. To notify the user of the identification information, a sound announcing an in-plane state or an out-of-plane state of the puncture needle, or a predetermined sound indicating an in-plane state or an out-of-plane state of the puncture needle may be used. The in-plane or out-of-plane state of the puncture needle may be displayed on the display 16, and a predetermined symbol indicating the in-plane or out-of-plane state may be displayed on the display 16.

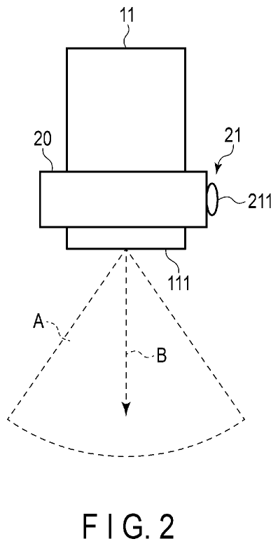

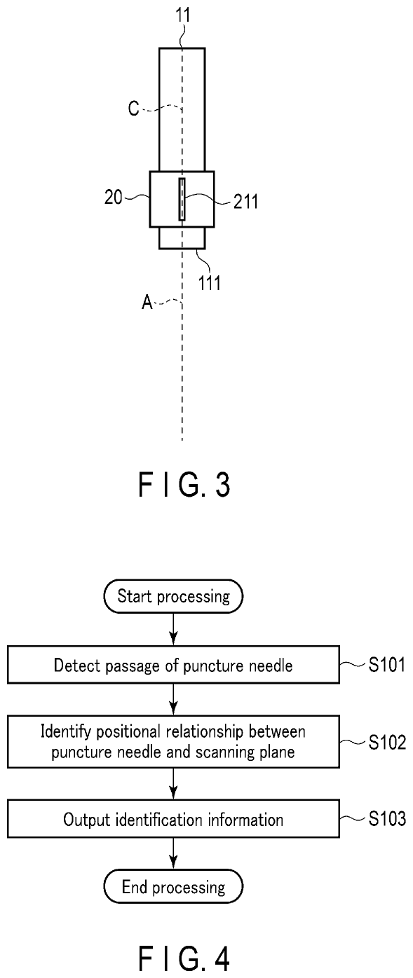

Next, an example of the configuration of the puncture guiding apparatus 20 is described with reference to FIGS. 2 and 3. The puncture guiding apparatus 20 is attached in the vicinity of the distal end portion of the ultrasonic probe 11. Herein, the direction parallel to the scanning plane A and orthogonal to the acoustic radiation direction B is referred to as a “scanning direction”, and the direction orthogonal to the scanning plane A is referred to a “short-axis direction”. The scanning direction may be referred to as a long-axis direction. FIG. 2 is a diagram viewed from one side of the short-axis direction, and FIG. 3 is a diagram viewed from one side of the scanning direction.

The detection unit 21 has a camera 211 for capturing an image of a detection range of the passage of the puncture needle N. The camera 211 is arranged in the vicinity of the living body contacting surface 111, facing outward with respect to the scanning direction of the ultrasonic probe 11, and is capable of capturing an image of a predetermined imaging range located outward in the scanning direction with respect to the camera 211. As shown in FIG. 3, the camera 211 is arranged along the acoustic radiation direction B on the puncture plane C including the scanning plane A. The camera 211 has one or more imaging sensors for the scanning direction A and two or more imaging sensors for the acoustic radiation direction B. The photographic range of the camera 211 may be set appropriately depending on the diameter of the puncture needle N, a permissible tilt angle of the puncture needle N with respect to the puncture plane C, and other values.

### (Operation)

Next, an operation of puncture guidance processing performed by the puncture guiding apparatus 20 of the ultrasonic diagnostic system 1 is described. The puncture guidance processing is a processing of assisting a user who performs a puncture technique with inserting the puncture needle accurately. FIG. 4 is a flowchart showing an example of the procedure of the puncture guidance processing. The processing procedure described below is merely an example, and the processing can be changed to the extent possible. Omission, replacement, or addition of a step in the process procedure described hereinafter can be made as appropriate, in accordance with an actual situation where the present embodiment is realized.

### (Puncture Guidance Processing)

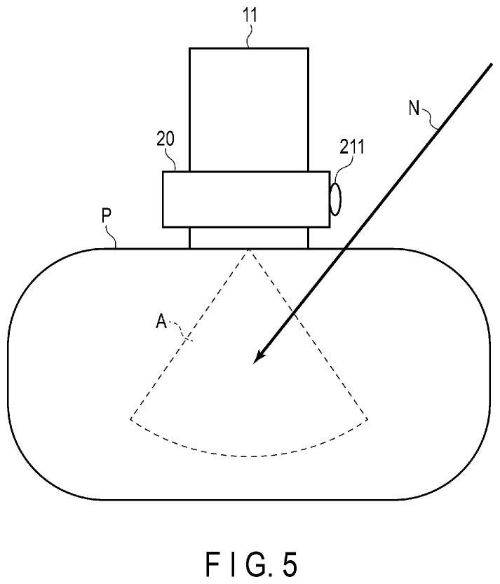

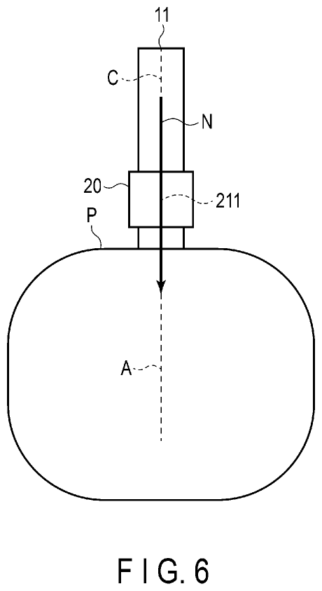

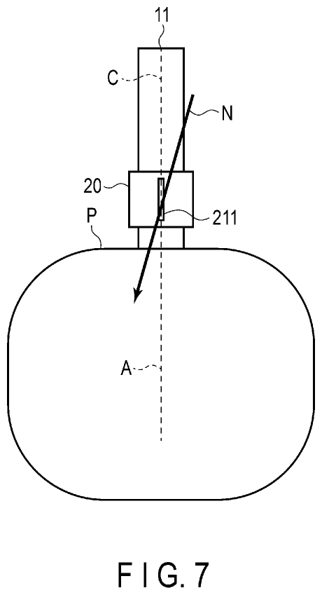

The puncture guidance processing is performed when the puncture needle is inserted into a subject P while capturing an image with the ultrasonic probe 11. The puncture guidance processing may be performed before the puncture needle N is inserted into the subject P or during insertion of the puncture needle N. The puncture guidance processing may commence upon a user's operation or in response to commencing of image capturing by the ultrasonic probe 11. FIG. 5 through FIG. 7 show the puncture with the puncture needle N. FIG. 5 shows the ultrasonic probe 11 viewed from one side in the short-axis direction, and FIGS. 6 and 7 show the ultrasonic probe 11 viewed from one side in the scanning direction.

### (Step S101)

After the puncture guidance processing commences, the detection unit 21 causes the camera 211 to start capturing an image, and detects passage of the puncture needle N in a range which is captured by the camera 211 based on the image captured by the camera 211. At this time, the detection unit 21 detects the puncture needle N in the captured image by performing a publicly known image processing technique for detecting a predetermined object in a captured image. The detection unit 21 determines that the puncture needle N has passed through the photographic range if the puncture needle N is detected in the captured image and that the puncture needle N did not pass through the photographic range if the puncture needle N is not detected in the captured image. The processing of detecting passage of the puncture needle N may be performed at predetermined time intervals, at all times, or at a timing at which the user inputs an operation. The detection unit 21 successively transmits a determination result to the identification information output unit 22 every time a determination regarding passing/not passing of the puncture needle N is made.

### (Step S102)

If passage of the puncture needle N is detected, the identification information output unit 22 identifies a positional relationship between the puncture needle N and the scanning plane A. At this time, the identification information output unit 22 determines whether or not the puncture needle N is located in the puncture plane C based on a detection result of the puncture needle N obtained from each image capturing element of the camera 211.

For example, as shown in FIG. 6, if the puncture needle N is detected at all positions in the acoustic radiation direction B, the identification information output unit 22 determines that the puncture needle N is located in the puncture plane C and the puncture needle N is in plane. As shown in FIG. 7, on the other hand, if there is a position at which the puncture needle N is not detected in the acoustic radiation direction B, the identification information output unit 22 determines that the puncture needle N is tilted with respect to the puncture plane C and the puncture needle N is not in plane.

### (Step S103)

The identification information output unit 22 outputs identification information based on a positional relationship between the puncture needle N and the puncture plane C, which was identified in the processing in step S102. At this time, the identification information output unit 22 notifies the technician of information, as identification information, indicating whether the puncture needle N is in plane or out of plane. For example, the identification information output unit 22 notifies the technician of an in-plane or out-of-plane state of the puncture needle N as identification information, using text or sound. The identification information output unit 22 may notify the technician only when the puncture needle N is in plane or only when the puncture needle N is out of plane. The technician checks whether or not the puncture needle N is in plane or out of plane, so that they can proceed with the puncture technique, with the puncture needle N being in an in-plane state.

Hereinafter, advantageous effects of the ultrasonic diagnostic system 1 having the puncture guiding apparatus 20 according to the present embodiment are described.

The ultrasonic diagnostic system 1 having a puncture guiding apparatus 20 of the present embodiment includes an ultrasonic probe 11 and a puncture guiding apparatus 20. The puncture guiding apparatus 20 includes a detection unit 21 and an identification information output unit 22. The detection unit 21 is arranged in the vicinity of a living body contacting surface 111 of the ultrasonic probe 11 that is brought into contact with a living body, and detects passage of a puncture needle. The identification information output unit 22 outputs identification information for identifying a positional relationship between the puncture needle and the scanning plane of the ultrasonic probe 11 based on a detection result obtained by the detection unit 21.

In the present embodiment, the detection unit 21 includes a camera 211. The camera 211 is arranged on the puncture plane including the scanning plane, and detects passage of the puncture needle within the puncture plane. The identification information output unit 22 outputs identification information identifying whether the puncture needle is located in the puncture plane. Specifically, the identification information output unit 22 identifies whether or not the puncture needle is in or not in the puncture plane, and notifies a technician of the identification result using sound or a screen display.

According to the above configuration, a technician who performs a puncture technique can easily know the position of the puncture needle with respect to the scanning plane by checking the identification information for identifying a positional relationship between the puncture needle and the scanning plane of the ultrasonic probe 11, and can accurately puncture a target area located within the scanning plane. Since a target area can be accurately punctured without using a puncture adapter, etc., the puncture needle can be inserted while being in the scanning plane, without a need to replace a puncture guide depending on a diameter of a puncture needle. Furthermore, it is possible to puncture a target area accurately, without a need to attach a special device to the puncture needle for locating the position of the puncture needle.

### First Modification

Next, a first modification of the first embodiment will be described. The present modification is a modification of the configuration of the first embodiment as will be described below. Descriptions of the configurations, operations, and advantageous effects similar to those of the first embodiment will be omitted.

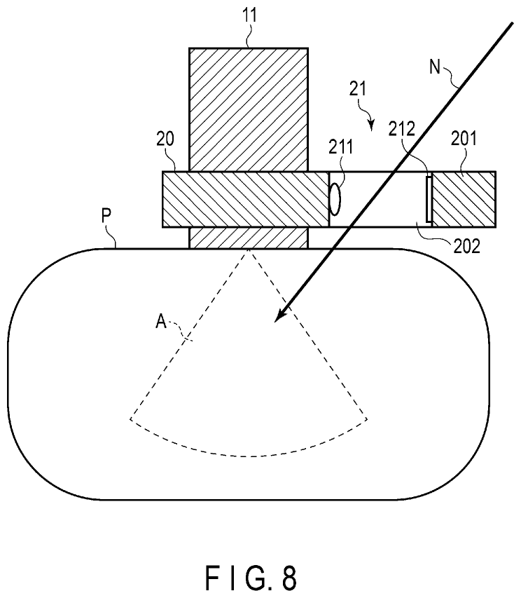

FIG. 8 is a diagram showing a configuration of the puncture guiding apparatus 20 of the present modification. FIG. 8 shows a cross section passing through the scanning plane A. As shown in FIG. 8, the puncture guiding apparatus 20 includes a projection 201 projecting outward along the scanning direction and an insertion hole 202 into which the puncture needle N is inserted. The insertion hole 202 penetrates through the projection 201 along the acoustic radiation direction. The projection 201 and the insertion hole 202 are respectively provided at positions at which the puncture plane including the scanning plane A passes through. The camera 211 is attached on the surface of the inner wall of the insertion hole 202, facing outward with respect to the scanning direction.

The detection unit 21 has an identification marker 212 in addition to the camera 211. The identification marker 212 is attached to the inner wall of the insertion hole 202, facing inward with respect to the scanning direction, and is arranged at a position facing the camera 211. The identification marker 212 is a sticker of a color differing from the color of the puncture needle N, for example.

The detection unit 21 determines that the puncture needle N is present at a position at which the identification marker 212 is not pictured. At this time, since the surroundings are not pictured in the image captured by the camera 211 and only the puncture needle N and the identification marker 212 of which the color is recorded in advance are pictured, it is easy to identify the puncture needle N, and the accuracy of detecting passage of the puncture needle N is improved.

The arrangement of the camera 211 and the identification marker 212 may be reversed. For example, the identification marker 212 may be arranged on the outward-facing surface and the camera 211 may be arranged on the inward-facing surface.

### Second Modification

Next, a second modification of the first embodiment will be described. The present modification is a modification of the configuration of the first embodiment as will be described below. Descriptions of the configurations, operations, and advantageous effects similar to those of the first embodiment will be omitted.

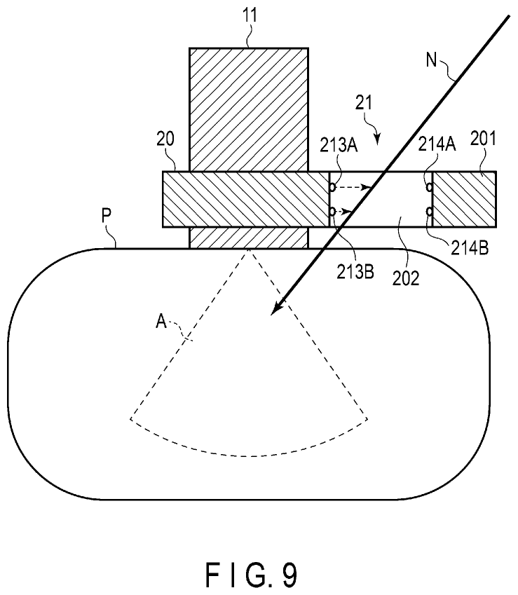

FIG. 9 is a diagram showing the configuration of the puncture guiding apparatus 20 according to the second modification. FIG. 9 shows a cross section passing through the scanning plane A. In the second modification, the detection unit 21 has an optical sensor for detecting passage of the puncture needle. An example of the optical sensor is an infrared sensor. In the second modification, the optical sensor has two transmission units (transmitters) 213A and 213B and two reception units (receptors) 214A and 214B. Similar to the structure shown in FIG. 8, the puncture guiding apparatus 20 has a projection 201 projecting outward along the scanning direction and an insertion hole 202 into which the puncture needle N is inserted.

The transmission units 213A and 213B are arranged on the inner wall of the insertion hole 202, facing outward with respect to the scanning direction. The reception units 214A and 214B are arranged on the inner wall of the insertion hole 202, facing inward with respect to the scanning direction and facing the transmission units 213A and 213B. The transmission units 213A and 213B and the reception units 214A and 214B are arranged at positions at which the puncture plane including the scanning plane A passes through. The transmission unit 213A and the reception unit 214A are arranged at approximately the same position with respect to the acoustic radiation direction B, and the transmission unit 213B and the reception unit 214B are arranged at approximately the same position with respect to the acoustic radiation direction B. The transmission units 213A and the transmission unit 213B are arranged at different positions with respect to the acoustic radiation direction B, and the reception unit 214A and the reception unit 214B are arranged at approximately the same position with respect to the acoustic radiation direction B.

The transmission unit 213A transmits light toward the reception unit 214A, and the reception unit 214A receives light transmitted from the transmission unit 213A. The transmission unit 213B transmits light toward the reception unit 214B, and the reception unit 214B receives light transmitted from the transmission unit 213B.

The detection unit 21 detects passage of the puncture needle N in the insertion hole 202. At this time, if light is received by the reception unit 214A or 214B, the detection unit 21 determines that the puncture needle N passes through the insertion hole 202, and if light is not received by the reception unit 214A or 214B, the detection unit 21 determines that the puncture needle N has not passed through the insertion hole 202.

The identification information output unit 22 identifies a positional relationship between the puncture needle N and the scanning plane A if passage of the puncture needle N is detected. At this time, the identification information output unit 22 determines whether or not the puncture needle N is located on the puncture plane including the scanning plane A based on a result of detection of light at the reception unit 214A or 214B.

For example, if light is received by both of the reception units 214A and 214B, the identification information output unit 22 determines that the puncture needle N is located on the puncture plane and is in plane.

If light is received by neither the reception unit 214A nor the reception unit 214B, the identification information output unit 22 determines that the puncture needle N is tilted with respect to the puncture plane C and not in plane.

Hereinafter, the ultrasonic diagnostic system 1 according to the second modification is described below.

In the second modification, the ultrasonic diagnostic system 1 has transmission units 213A and 213B and reception units 214A and 214B as optical sensors, and the transmission units 213A and 213B and the reception units 214A and 214B are arranged on the puncture plane including the scanning plane. The detection unit 21 detects passage of a puncture needle within the puncture plane based on a reception result at the reception unit 214A or 214B, and the identification information output unit 22 identifies whether the puncture needle is in or not in the puncture plane based on a reception result at the reception unit 214A or 214B, and notifies a technician of the information.

According to the above configuration, similar to the first embodiment, a technician who performs a puncture technique can easily know the position of the puncture needle with respect to the scanning plane by checking the identification information for identifying a positional relationship between the puncture needle and the scanning plane of the ultrasonic probe 11, and can accurately puncture a target area located in the scanning plane.

The arrangement of the transmission units 213A and 213B and the reception units 214A and 214B may be reversed. For example, the reception units 214A and 214B can be arranged on the outward-facing surface, and the transmission units 213A and 213B can be arranged on the inward-facing surface.

In the present modification, a combination of two sets of optical sensors (a set of the transmission unit 213A and the reception unit 214A and a set of the transmission unit 213B and the reception unit 214B) has been explained; however, accuracy of puncture needle detection can be improved by using three or more sets of optical sensors.

### Third Modification

Next, a third modification of the first embodiment will be described. The present modification is a modification of the configuration of the first embodiment as will be described below. Descriptions of the configurations, operations, and advantageous effects similar to those of the first embodiment will be omitted.

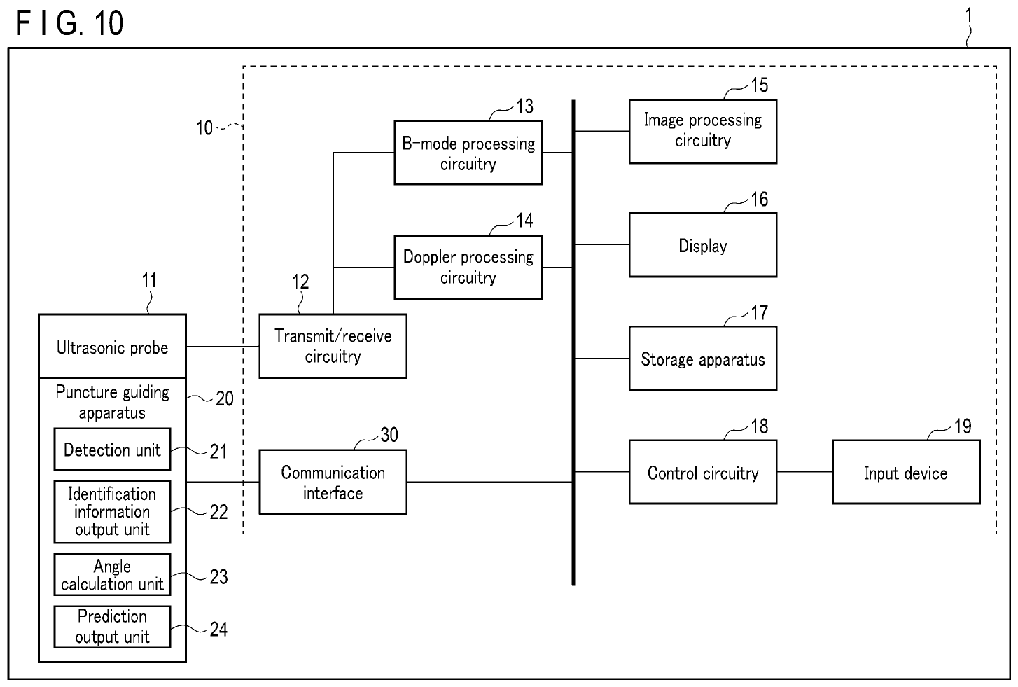

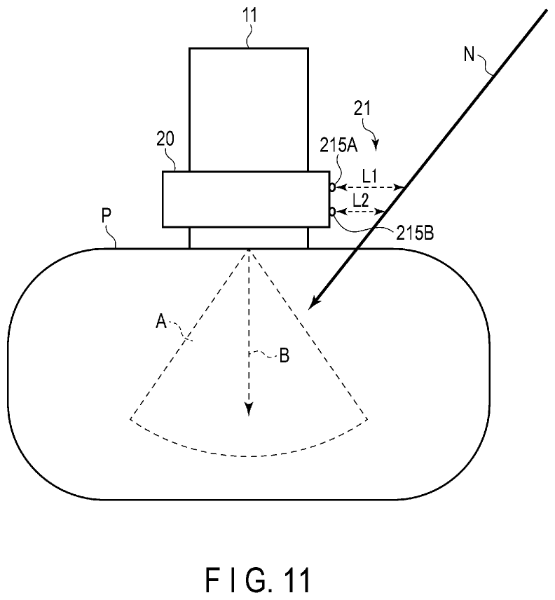

FIG. 10 is a diagram showing an example configuration of a puncture guiding apparatus 20 according to the third modification. FIG. 11 is a diagram showing a configuration of the puncture guiding apparatus 20 of the third modification. FIG. 11 is a diagram of the puncture guiding apparatus 20, viewed from one side with respect to a short-axis direction.

As shown in FIG. 11, the detection unit 21 of the puncture guiding apparatus 20 has two cameras 215A and 215B. The cameras 215A and 215B are arranged at positions at which the puncture plane including the scanning plane A passes through. The cameras 215A and 215B are arranged at different positions with respect to the acoustic radiation direction B. The detection unit 21 has an auto-focus function for automatically focusing the cameras 215A and 215B on the puncture needle N.

As shown in FIG. 10, the puncture guiding apparatus 20 further includes an angle calculation unit 23 and a prediction output unit 24. The angle calculation unit 23 and the prediction output unit 24 are realized by a processor for analyzing a signal from a sensor by executing a program stored in a storage apparatus (not shown) that is installed on the puncture guiding apparatus 20.

The angle calculation unit 23 obtains focus positions of the cameras 215A and 215B from the detection unit 21, and calculates an angle of the puncture needle N with respect to the acoustic radiation direction B (hereinafter, an “insertion angle”) based on the obtained focus positions. The insertion angle may be called a “puncture angle”.

The prediction output unit 24 predicts the position at which the puncture needle N passes through (hereinafter a “prediction line”) if the puncture needle N advances to the scanning plane A along a straight line connecting the focus positions of the cameras 215A and 215B based on the insertion angle of the puncture needle N calculated by the angle calculation unit 23, and outputs the prediction line. For example, the prediction output unit 24 acquires an ultrasound image from the image processing circuitry 15, generates a fusion image in which a prediction line is superimposed on an ultrasound image, and causes the display 16 to display the generated fusion image. The prediction output unit 24 may cause the display 16 to display the insertion angle of the puncture needle N together with the ultrasound image.

### (Operation)

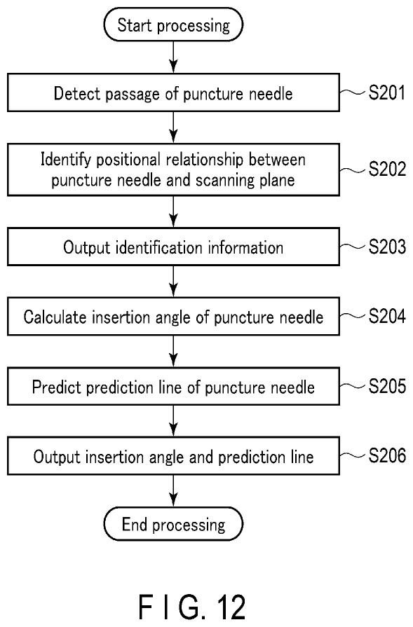

Next, an operation of puncture guidance processing performed by the puncture guiding apparatus 20 of the ultrasonic diagnostic system 1 of the present modification is described. FIG. 12 is a flowchart showing an example of the procedure of the puncture guidance processing of the present modification. The processing procedure described below is merely an example, and the processing can be changed to the extent possible. Omission, replacement, or addition of a step in the process procedure described hereinafter can be made as appropriate, in accordance with an actual situation where the present embodiment is realized.

### (Puncture Guidance Processing)

### (Step S201)

After the puncture guidance processing commences, similar to step S101 shown in FIG. 4, the detection unit 21 causes the cameras 215A and 215B to start capturing images, detects the puncture needle N from the captured images based on the captured images acquired by the cameras 215A and 215B, and detects passage of the puncture needle N in the photographic ranges of the cameras 215A and 215B. At this time, the detection unit 21 implements the auto-focus function to focus the cameras 215A and 215B on the detected puncture needle N. The detection unit 21 transmits, to the angle calculation unit 23, the focal distances L1 and L2 of the cameras 215A and 215B at the timing when the puncture needle N is detected.

### (Step S202)

Similar to step S102 of FIG. 4, the identification information output unit 22 determines whether or not the puncture needle N is located on the puncture plane C based on a detection result of the puncture needle N acquired from the cameras 215A and 215B, and identifies a positional relationship between the puncture needle N and the scanning plane A.

### (Step S203)

Similar to step S103 of FIG. 4, the identification information output unit 22 notifies the technician of information, as identification information, indicating whether the puncture needle N is in plane or not in plane.

### (Step S204)

The angle calculation unit 23 calculates, if the puncture needle N is in plane, an insertion angle of the puncture needle N with respect to the acoustic radiation direction B based on the focal distances L1 and L2 of the cameras 215A and 215B. At this time, the angle calculation unit 23 calculates a straight line connecting the focus positions of the cameras 215A and 215B, using the focal distances L1 and L2 of the cameras 215A and 215B, and calculates the angle of the straight line with respect to the acoustic radiation direction B as the insertion angle of the puncture needle N.

### (Step S205)

The prediction output unit 24 estimates a prediction line indicating a position at which the puncture needle N passes through if the puncture needle N is inserted into the scanning plane A, based on the insertion angle of the puncture needle N. At this time, the prediction output unit 24 calculates a prediction line indicating the position of the puncture needle N if the puncture needle N is inserted into the scanning plane A along an extended line of the straight line connecting the focus positions of the cameras 215A and 215B.

### (Step S206)

The prediction output unit 24 acquires an ultrasound image from the image processing circuitry 15, generates a fusion image obtained by superimposing the prediction line on the ultrasound image, and causes the display 16 to display the generated fusion image and the insertion angle.

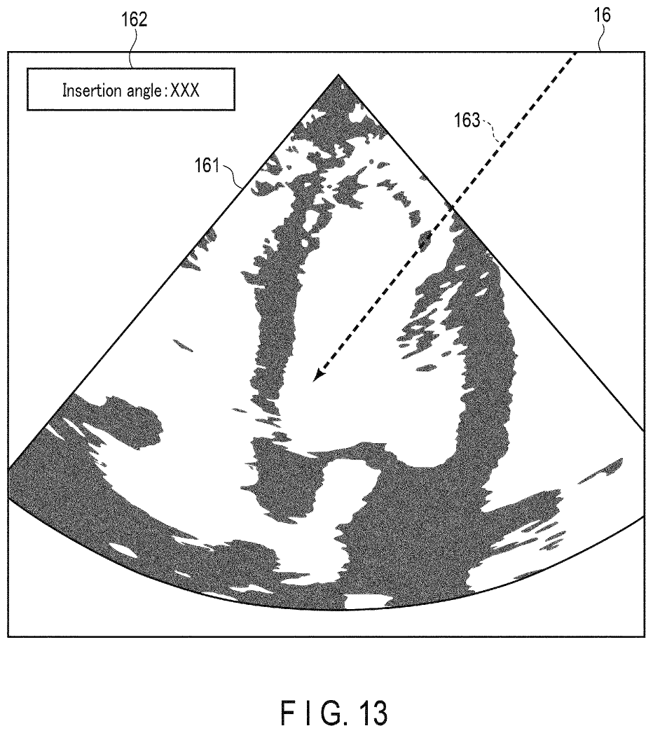

FIG. 13 is an example of a fusion image displayed on the display 16. FIG. 13 shows that the image display area 161 for displaying a fusion image and the insertion angle display area 162 are provided on the display screen of the display 16. In the image display area 161, the prediction line 163 is superimposed on the ultrasound image. In the insertion angle display area 162, the insertion angle of the puncture needle N with respect to the acoustic radiation direction B is displayed.

Hereinafter, advantageous effects of the ultrasonic diagnostic system 1 having the puncture guiding apparatus 20 according to the third modification are described.

The puncture guiding apparatus 20 of the present modification further includes an angle calculation unit 23 and a prediction output unit 24. The detection unit 21 includes cameras 215A and 215B and has a focus function for focusing the cameras 215A and 215B on the puncture needle. The angle calculation unit 23 calculates the insertion angle of the puncture needle with respect to the puncture plane including the scanning plane based on the focus positions of the cameras 215A and 215B. The prediction output unit 24 predicts and outputs a prediction line through which the puncture needle passes when the puncture needle advances to the scanning plane based on the insertion angle. For example, the prediction line of the puncture needle can be superimposed on the ultrasound image displayed on the display 16 and displayed along with the insertion angle.

According to the above-described configuration, a technician who performs a puncture technique can easily check if they can puncture a target area at a current insertion angle of a puncture needle by checking the current insertion angle and a prediction line if the puncture needle is inserted into the scanning plane at the current insertion angle. For example, if the prediction line does not overlap the target area, the technician can accurately puncture the target area by adjusting the insertion angle of the puncture needle while checking the prediction line.

In the third modification, an example where two cameras 215A and 215B are used is explained; accuracy of calculation of an insertion angle of the puncture needle can be improved by using three or more cameras.

### Second Embodiment

Next, the second embodiment will be described. The present embodiment is a modification of the configuration of the first embodiment as will be described below. Descriptions of the configurations, operations, and advantageous effects similar to those of the first embodiment will be omitted. The ultrasonic diagnostic system 1 of the first embodiment is used mainly with a technique of inserting a puncture needle along a scanning direction (long-axis direction) of the ultrasonic probe 11. On the other hand, the ultrasonic diagnostic system 1 of the present embodiment is used mainly with a technique of inserting a puncture needle along a short-axis direction of the ultrasonic probe 11, as with a central venous puncture.

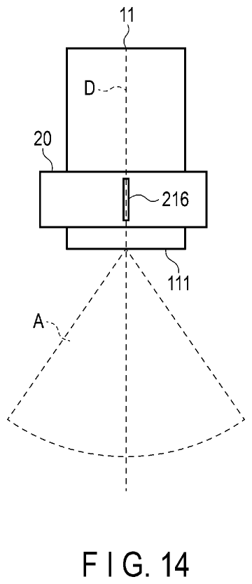

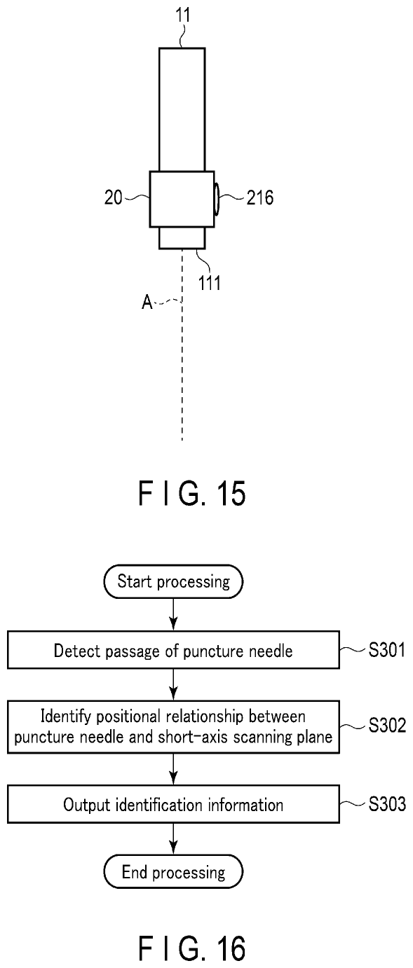

An example of the configuration of the puncture guiding apparatus 20 is described with reference to FIGS. 14 and 15. FIG. 14 is a diagram viewed from one side of the short-axis direction, and FIG. 15 is a diagram viewed from one side of the scanning direction.

The detection unit 21 is provided at a position at which a positional relationship of the puncture needle N with respect to the puncture plane D can be determined. The puncture plane D in the present embodiment is a plane intersecting with the scanning plane A. Specifically, the puncture plane D is a plane approximately perpendicular to the scanning plane A. The puncture plane D is typically set to a central position of the ultrasonic probe 11 with respect to the scanning direction. The puncture plane D may be set to a position away from the central position of the ultrasonic probe 11 with respect to the scanning direction.

In the present embodiment, an example wherein the detection unit 21 has a camera 216 will be explained; however, an optical sensor or an ultrasonic sensor may be used instead of the camera 216, similarly to the first embodiment.

The camera 216 is arranged in the vicinity of the living body contacting surface 111, facing outward with respect to the short-axis direction of the ultrasonic probe 11, and is capable of capturing an image of a predetermined imaging range located outward in the short-axis direction with respect to the camera 216. As shown in FIG. 14, the camera 216 is arranged along the acoustic radiation direction B on the puncture plane D. The camera 216 has one or more imaging sensors for the short-axis direction A and two or more imaging sensors for the acoustic radiation direction B. The photographic range of the camera 216 may be set appropriately dependent on the diameter of the puncture needle N, a permissible tilt angle of the puncture needle N with respect to the puncture plane D, and other values.

The identification information output unit 22 outputs identification information for identifying a positional relationship between the puncture needle N and the puncture plane D based on a detection result of the detection unit 21. In the present embodiment, the puncture plane D is a plane intersecting with the scanning plane A of the ultrasonic probe 11. For this reason, the identification information output unit 22 identifies a positional relationship between the puncture needle N and the plane intersecting with the scanning plane A of the ultrasonic probe 11. At this time, the identification information output unit 22 determines, based on the detection result obtained from the detection unit 21, whether the puncture needle N is in or not in the puncture plane D.

### (Operation)

Next, an operation of puncture guidance processing performed by the puncture guiding apparatus 20 of the ultrasonic diagnostic system 1 is described. FIG. 16 is a flowchart showing an example of the procedure of the puncture guidance processing. The processing procedure described below is merely an example, and the processing can be changed to the extent possible. Omission, replacement, or addition of a step in the process procedure described hereinafter can be made as appropriate, in accordance with an actual situation where the present embodiment is realized.

### (Puncture Guidance Processing)

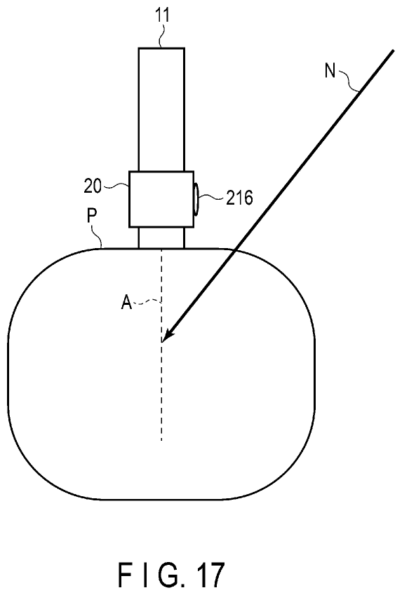

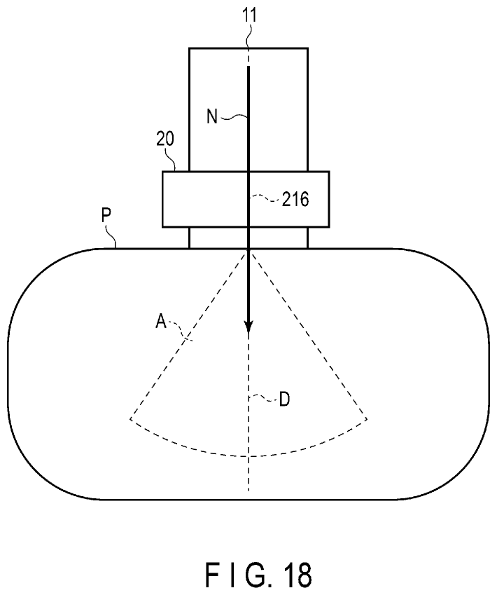

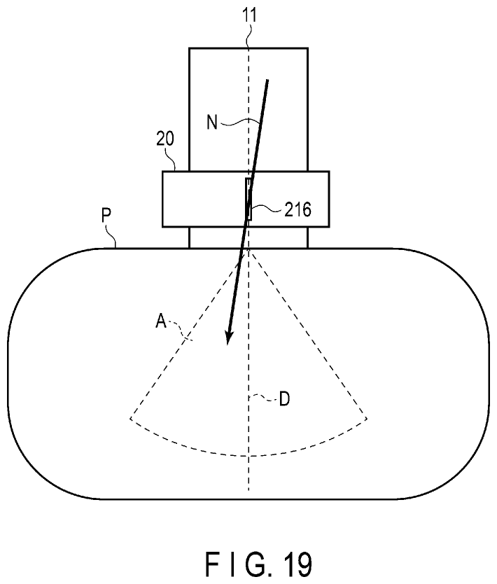

FIG. 17 through FIG. 19 show the puncture with the puncture needle N. FIG. 17 shows the ultrasonic probe 11 viewed from one side in the scanning direction, and FIGS. 18 and 19 show the ultrasonic probe 11 viewed from one side in the short-axis direction.

### (Step S301)

After the puncture guidance processing is commenced, the detection unit 21 causes the camera 216 to start capturing an image, and detects passage of the puncture needle N in the photographic range of the camera 216 based on the image captured by the camera 216, similar to step S101 of the first embodiment. In the present embodiment, the photographic range of the camera 216 is in plane with a plane approximately perpendicular to the scanning plane A.

### (Step S302)

The identification information output unit 22 identifies a positional relationship between the puncture needle N and the scanning plane D in the short-axis direction if passage of the puncture needle N is detected.

At this time, the identification information output unit 22 determines whether or not the puncture needle N is located on the puncture plane D based on a detection result of the puncture needle N obtained from each image capturing element of the camera 216.

For example, as shown in FIG. 18, if the puncture needle N is detected at all positions in the acoustic radiation direction B, the identification information output unit 22 determines that the puncture needle N is located on the puncture plane D and that the puncture needle N is in plane. As shown in FIG. 19, on the other hand, if there is a position at which the puncture needle N is not detected on the acoustic radiation direction B, the identification information output unit 22 determines that the puncture needle N is tilted with respect to the puncture plane D and that the puncture needle N is not in plane.

### (Step S303)

The identification information output unit 22 outputs identification information based on a positional relationship between the puncture needle N and the puncture plane D. At this time, similar to step S103 of the first embodiment, the identification information output unit 22 notifies the technician of information, as identification information, indicating an in-plane state or an out-of-plane state of the puncture needle N.

Hereinafter, advantageous effects of the ultrasonic diagnostic system 1 having the puncture guiding apparatus 20 according to the present embodiment are described.

In the present embodiment, the identification information output unit 22 outputs identification information for identifying a positional relationship between the puncture needle and the plane intersecting with the scanning plane of the ultrasonic probe 11 based on a detection result obtained by the detection unit 21. The detection unit 21 has the camera 216, and the camera 216 is arranged within the plane intersecting with the scanning plane to detect passage of the puncture needle within the puncture plane.

According to the above-described configuration, it is possible to obtain advantageous effects similar to those of the first embodiment. More specifically, a technician who performs a puncture technique can easily know the position of the puncture needle with respect to the puncture plane by checking the identification information for identifying a positional relationship between the puncture needle and the puncture plane, and can therefore accurately puncture a target area located in the puncture plane. Since a target area can be accurately punctured without using a puncture adapter, etc., the puncture needle can be inserted while being in the puncture plane, without a need to replace a puncture guide dependent on a diameter of a puncture needle.

### Modifications of Embodiment

In the second embodiment, an example wherein a sensor of the detection unit 21 is arranged on the puncture plane to detect passage of the puncture needle within the puncture plane and whether or not the puncture needle is in plane or not in plane is thereby identified is explained; however, the embodiment is not limited to this example.

As another example, a sensor of the detection unit 21 may be arranged on the puncture plane to detect passage of the puncture needle outside the puncture plane and whether or not the puncture needle is in plane or not in plane may be identified. For example, if the puncture plane is a plane including the scanning plane, the detection unit 21 is arranged on a plane including the scanning plane, and detects passage of the puncture needle outside the plane including the scanning plane. The detection unit 21 is arranged within a certain range in the vicinity of the scanning plane. The identification information output unit 22 outputs identification information identifying whether or not at least a part of the puncture needle is located in the plane including the scanning plane. At this time, if the puncture needle is detected by the detection unit 21, the identification information output unit 22 determines that the puncture needle is not in plane, and if the puncture needle is not detected by the detection unit 21, the identification information output unit 22 determines that the puncture needle is in plane.

In the foregoing embodiments, the puncture guiding apparatus 20 is an external adapter that can be detachably attached to the ultrasonic probe 11, and the example wherein the detection unit 21, the identification information output unit 22, the angle calculation unit 23, and the prediction output unit 24, etc. may be detachably attached to the ultrasonic probe 11 was explained; however, each of these components of the puncture guiding apparatus 20 may be integrated with the ultrasonic probe 11. For example, a sensor of the detection unit 21 may be provided in the vicinity of the living body contacting surface 111 of the ultrasonic probe 11 on the outer surface, and the identification information output unit 22, the angle calculation unit 23, and the prediction output unit 24, etc. may be mounted inside the ultrasonic probe 11.

The hardware configuration of the ultrasonic diagnostic system 1 is not limited to the configurations described in the foregoing embodiments. For example, at least a part of the transmit/receive circuitry 12, the B-mode processing circuitry 13, the Doppler processing circuitry 14, the image processing circuitry 15, the display 16, the storage apparatus 17, the control circuitry 18, the input device 19, the identification information output unit 22, the angle calculation unit 23, and the prediction output unit 24 may be mounted in the ultrasonic probe 11. At least a part of the image processing circuitry 15, the display 16, the storage apparatus 17, the identification information output unit 22, the angle calculation unit 23, and the prediction output unit 24 may be mounted in a computer connected to the apparatus main body 10 via a network. At least a part of the identification information output unit 22, the angle calculation unit 23, or the prediction output unit 24 may be mounted in the apparatus main body 10. The control circuitry 18, the identification information output unit 22, the angle calculation unit 23, and the prediction output unit 24 are not necessarily mounted in separate hardware components and may be mounted in a single hardware component.

The term “processor” used in the above explanation means, for example, circuitry such as a CPU (central processing unit), a GPU (graphics processing unit), an ASIC (application specific integrated circuit), or a programmable logic device (for example, an SPLD (simple programmable logic device), a CPLD (complex programmable logic device), or an FPGA (field programmable gate array)). If the processor is a CPU, for example, the processor realizes its function by reading and executing the program stored in the storage circuitry. If the processor is for example an ASIC, on the other hand, the function is directly implemented in a circuit of the processor as a logic circuit, instead of storing a program in a storage circuit. Each processor of the present embodiment is not limited to a case where each processor is configured as a single circuit; a plurality of independent circuits may be combined into one processor to realize the function of the processor. Furthermore, a plurality of constituent elements shown in FIG. 1 or 10 may be integrated into one processor to implement the functions. The above description of the “processor” is applicable to the subsequent embodiments and modifications.

According to at least one of the foregoing embodiments, a puncture needle can be accurately inserted in a puncture technique using an ultrasonic probe and a puncture needle.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A puncture guiding apparatus comprising:
a detector configured to detect passage of a puncture needle, the detector arranged in a vicinity of a living body contacting surface of an ultrasonic probe that is brought into contact with a living body; and
processing circuitry configured to output identification information for identifying a positional relationship between the puncture needle and a scanning plane of the ultrasonic probe based on a detection result obtained by the detector.

2. The puncture guiding apparatus of claim 1, wherein
the processing circuitry is configured to output identifying information for identifying whether or not the puncture needle is located in a plane including the scanning plane.

3. The puncture guiding apparatus of claim 2, wherein
the detector detects passage of the puncture needle in the plane including the scanning plane, the detector arranged on the plane including the scanning plane.

4. The puncture guiding apparatus of claim 1, wherein
the processing circuitry is configured to output identifying information for identifying whether or not at least a part of the puncture needle is located out of the plane including the scanning plane.

5. The puncture guiding apparatus of claim 4, wherein
the detector is arranged outside the plane including the scanning plane, and detects passage of the puncture needle outside the plane including the scanning plane.

6. The puncture guiding apparatus of claim 1, wherein
the detector has a camera for capturing an image of a range in which passage of the puncture needle is detected.

7. The puncture guiding apparatus of claim 1, wherein
the detector has an optical sensor for detecting passage of the puncture needle.

8. The puncture guiding apparatus of claim 7, wherein
the optical sensor includes a transmitter configured to transmit light and a receptor configured to receive the transmitted light, the transmission unit and the receptor arranged in the range in which passage is detected.

9. The puncture guiding apparatus of claim 1, wherein
the detector and the processing circuitry are integrated with the ultrasonic probe.

10. The puncture guiding apparatus of claim 1, wherein
the detector and the processing circuitry are detachably attached to the ultrasonic probe.

11. The puncture guiding apparatus of claim 6, wherein
the detector is configured to focus the camera on the puncture needle, and
the processing circuitry is configured to calculate an angle of the puncture needle with respect to an acoustic radiation direction of the ultrasonic probe based on a focus position.

12. The puncture guiding apparatus of claim 11, wherein
the processing circuitry predicts a path through which the puncture needle passes if the puncture needle advances to the scanning plane and outputs the predicted path and the angle.

13. An ultrasonic diagnostic system comprising:
the ultrasonic probe; and
the puncture guiding apparatus of claim 1.

14. A puncture guiding apparatus comprising:
a detector configured to detect passage of a puncture needle, the detector arranged in a vicinity of a living body contacting surface of an ultrasonic probe that is brought into contact with a living body; and
processing circuitry configured to output identification information for identifying a positional relationship between the puncture needle and a plane intersecting with a scanning plane of the ultrasonic probe based on a detection result obtained by the detector.

15. A puncture guiding apparatus comprising:
a detector configured to detect passage of a puncture needle, the detector arranged in a vicinity of a living body contacting surface of an ultrasonic probe that is brought into contact with a living body; and
processing circuitry configured to output identification information for identifying a positional relationship between the puncture needle and the puncture plane including a target position at which the puncture needle is slated to be inserted based on a detection result obtained by the detector.
